(19) United States
(12) Patent Application Publication
(10) Pub. No.: US 2025/0120992 A1
(43) Pub. Date: Apr. 17, 2025

(54) COMPOSITION FOR IMPROVING LIVER HEALTH COMPRISING GALLOCATECHIN GALLATE AND ISOQUERCITRIN

(71) Applicant: AMOREPACIFIC CORPORATION, Seoul (KR)

(72) Inventors: Hyun Woo JEONG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/823,345

(22) Filed: Sep. 3, 2024

(30) Foreign Application Priority Data
Oct. 11, 2023 (KR) 10-2023-0135342

(51) Int. Cl. A61K 31/7048; A61K 31/353; A61P 1/16

(52) U.S. Cl. CPC A61K 31/7048; A61K 31/353; A61P 1/16

(57) ABSTRACT

This specification discloses a method for improving liver health, including administering an effective amount of Gallocatechin Gallate (GCG) and Isoquercitrin (IQC) to a subject in need thereof. In an aspect, a composition including a complex component of Gallocatechin Gallate and Isoquercitrin effectively reduces oxidative stress and chronic inflammatory reaction that occurs during accumulation of lipids in liver tissue, thereby making it possible to enhance liver health and prevent, improve, or treat various liver diseases such as hepatitis, cirrhosis, and liver cancer.

### CROSS REFERENCE TO RELATED APPLICATION

The present application claims the benefit of priority to Korean Patent Application No. 10-2023-0135342, filed on Oct. 11, 2023, the entire contents of which are hereby incorporated by this reference.

### FIELD OF THE INVENTION

The present specification discloses a composition for improving liver health.

### BACKGROUND

Liver cells play an important role in regulating energy homeostasis in our body. Excess energy introduced through meals, etc., is stored in the form of a polymer complex such as glucose as glycogen and fatty acids as neutral lipids. When energy is insufficient due to an empty stomach, etc., the glycogen and the neutral lipids are decomposed with stored energy sources, and sent to tissues that need nutrients so that they can be used. However, in case the energy homeostasis is broken due to a larger amount of nutritional supplement compared to energy consumption, the excess energy is stored in fat cells. If the fat cells exceed their storage limit, a neutral fat in addition to the fat is accumulated in other tissues such as a liver and muscles. In particular, in case the fat is accumulated in the liver cells and accounts for more than 5% of the normal liver, it is called a fatty liver, which causes various liver diseases such as hepatitis, cirrhosis, and liver cancer due to oxidative stress and chronic inflammatory reaction that occurs during accumulation of lipids in the liver tissue. Because the liver performs many important roles which are necessary for survival, if liver health deteriorates, the metabolism of our entire body changes, which may be life-threatening. In particular, the liver is called a ‘silent organ’, and even if the liver is damaged to some extent, it is not easy to notice abnormal symptoms to make it difficult to recognize them, thereby also acting as an obstacle to managing the liver health. Moreover, in the case of fatty liver, since a specialized treatment targeting the fatty liver has not yet been developed, until now, the fatty liver should be managed through indirect treatment with treatment of lipid metabolism abnormalities such as hyperlipidemia (e.g., fibrate, statin, etc.) or through prevention by improvement of diet/lifestyle. Accordingly, there is an urgent need to develop targeted treatments or health functional foods that can improve liver health and prevent or treat the fatty liver and the liver disease.

### SUMMARY OF THE INVENTION

A purpose of the present disclosure is to provide a composition for improving liver health.

In order to achieve the above purpose, in an aspect, the present disclosure provides a composition for improving liver health, comprising Gallocatechin Gallate (GCG) and Isoquercitrin (IQC) as an active ingredient.

In an aspect, the composition of the present disclosure comprises a complex component of Gallocatechin Gallate and Isoquercitrin to effectively reduce oxidative stress and chronic inflammatory reaction that occurs during accumulation of lipids in the liver tissue, thereby making it possible to enhance liver health and prevent, improve, or treat various liver diseases such as hepatitis, cirrhosis, and liver cancer.

### BRIEF DESCRIPTION OF THE DRAWINGS

FIG. 1 compares an effect of improving non-alcoholic fatty liver disease according to a composition ratio of Gallocatechin Gallate (GCG) and Isoquercitrin (IQC) mixture (Adiphenon).

FIG. 2 compares an effect of improving alcoholic fatty liver disease according to a composition ratio of Adiphenon.

FIG. 3 compares an effect of inhibiting generation of active oxygen in liver cells according to a composition ratio of Adiphenon.

FIGS. 4 and 5 compare an effect of inhibiting inflammatory response-mediated gene expression in liver cells according to a composition ratio of Adiphenon.

FIG. 6 compares an effect of inhibiting accumulation of lipids in liver cells according to a detailed ratio of APGQ.

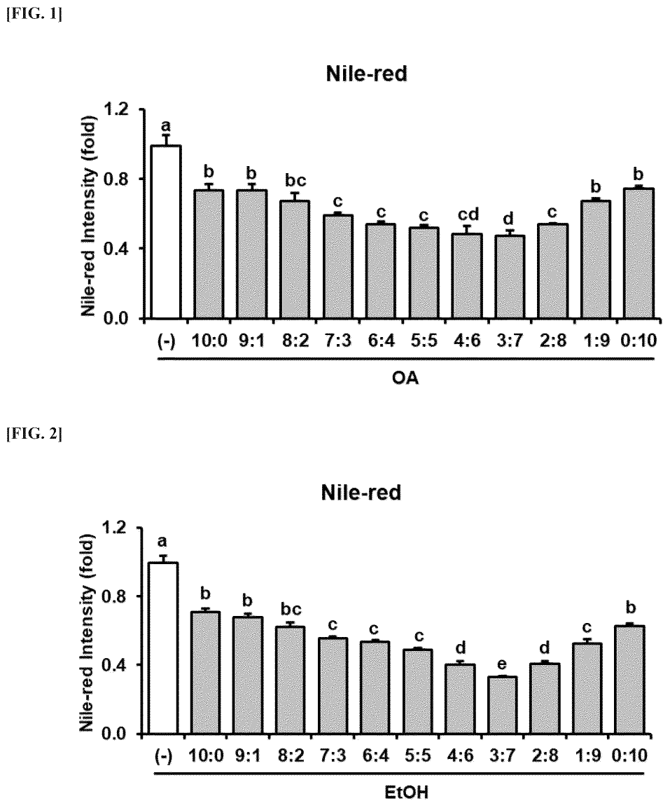

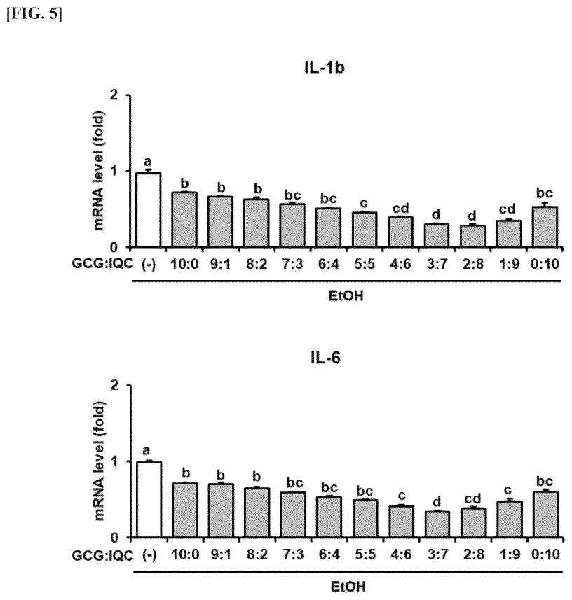

In FIGS. 1 to 6, experimental groups showing statistically significant differences (P<0.05) are indicated with different symbols.

### DETAILED DESCRIPTION OF THE INVENTION

Hereinafter, the present disclosure will be described in detail.

As used herein, the term “prevention” refers to any action that suppresses or delays a liver disease by applying the present pharmaceutical composition. The term “treatment” refers to any action by which the symptoms of a subject suspected or affected by the liver disease are improved or beneficially changed by applying the present pharmaceutical composition.

As used herein, the term “improvement” refers to any action by which the liver disease is improved or beneficially changed by applying the present composition.

In an aspect, the present disclosure relates to a composition for improving liver health, comprising Gallocatechin Gallate (GCG) and Isoquercitrin (IQC) as an active ingredient.

In an exemplary embodiment, the composition may be a pharmaceutical composition for preventing or treating a liver disease.

In an exemplary embodiment, the composition may be a food composition for preventing or improving the liver disease.

In an exemplary embodiment, Gallocatechin Gallate and Isoquercitrin may be contained in a weight ratio of 9:1 or more, 8:2 or more, 7:3 or more, 6:4 or more, 5:5 or more, or 4:6 or more, and may be contained in a weight ratio of 1:9 or less or 2:8 or less. For example, Gallocatechin Gallate and Isoquercitrin may be contained in a weight ratio of 9:1 to 1:9, but is not limited thereto.

In an exemplary embodiment, Gallocatechin Gallate and Isoquercitrin may be contained in a weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, 1:2.3 or more, or 1:2.4 or more, and may be contained in a weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, or 1:2.7 or less. In case Gallocatechin Gallate and Isoquercitrin are contained in the above weight ratio, liver health can be effectively promoted. For example, Gallocatechin Gallate and the Isoquercitrin may be contained in a weight ratio of 1:1 to 1:9 or 1:1.5 to 1:4, but is not limited thereto.

In an exemplary embodiment, the liver disease may be one or more selected from the group consisting of hepatitis, fatty liver, cirrhosis, and liver cancer.

In an exemplary embodiment, the liver disease may be an alcoholic liver disease.

In an exemplary embodiment, the composition may be characterized by being administered to a subject in need of reducing oxidative stress or inflammation in the liver cells.

In an exemplary embodiment, the composition may be characterized by being administered to a subject in need of reducing expression of one or more genes selected from the group consisting of IL-1b, IL-6, and iNOS.

In an exemplary embodiment, the composition may be characterized by being administered to a subject in need of inhibiting lipid accumulation in the liver cells.

In an exemplary embodiment, a daily dosage of the active ingredient in the composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. An effect of improving liver health is excellent at the above dosage. If the dosage is less than the above range, the effect of improving liver health is minimal, and if the dosage is higher than the above range, a problem such as toxicity may occur. The above dosage may be administered once or in divided doses several times per a day. For example, it may be administered 2 to 24 times per a day, 1 to 2 times per 3 days, 1 to 6 times per a week, 1 to 10 times per 2 weeks, 1 to 15 times per 3 weeks, 1 to 3 times per 4 weeks, or 1 to 12 times per a year, but is not limited thereto.

In an exemplary embodiment, the pharmaceutical composition may be provided in any formulation suitable for topical administration. For example, it may be administered orally, transdermally, intravenously, intramuscularly, or by subcutaneous injection. As an example, the pharmaceutical composition may be an injection, a solution for external use on the skin, a suspension, an emulsion, a gel, a patch, or a spray, but is not limited thereto. The formulation may be easily prepared according to conventional methods well known in the relevant field, and may appropriately contain a surfactant, an excipient, a hydrating agent, an emulsification accelerator, a suspending agent, a salt or buffer for adjusting osmotic pressure, a colorant, a spice, a stabilizer, a preservative, a conserving agent, or other commercially available aids.

In an exemplary embodiment, the active ingredient of the pharmaceutical composition will vary depending on the subject's age, gender, weight, pathological condition and its severity, a route of administration, or judgment of the prescriber. An appropriate dosage which is used based on these factors may be determined within the level of a person skilled in the art.

In an exemplary embodiment, in case the composition is used as an additive for a health functional food, the composition may be added as it is or used together with other foods or food ingredients, and may be used appropriately according to conventional methods. A mixed amount of the active ingredients may be appropriately determined depending on each purpose of use, such as prevention, health, or treatment. A formulation of the health functional food may be in any form of powders, granules, pills, tablets or capsules as well as general foods or beverages.

In an exemplary embodiment, there is no particular limitation on the type of the health functional food, and examples of foods to which the composition can be added include meat, confectionery, noodles, gum, dairy products including ice cream, various soups, beverages, tea, drinks, alcoholic beverages, vitamin complexes, etc., and may include all foods in the conventional sense.

In an exemplary embodiment, in the preparation of the health functional food or beverage, the composition may be added in an amount of 15 parts by weight or less, preferably 10 parts by weight or less, based on 100 parts by weight of the raw material. However, in the case of long-term intake for the purpose of health and hygiene or health control, the above amount may be below said range.

In an exemplary embodiment, the beverages among the health functional foods may contain various flavoring agents or natural carbohydrates as additional ingredients like the typical beverages. The above-mentioned natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. A sweetening agent may include natural sweetening agents such as thaumatin and stevia extract or synthetic sweetening agents such as saccharin and aspartame. A ratio of the natural carbohydrates may be about 0.01 to 0.04 g, preferably about 0.02 to 0.03 g, based on 100 mL of the beverages according to the present disclosure, but are not limited thereto.

In an exemplary embodiment, in addition to the above, the health functional food according to the present disclosure may contain various nutrients, vitamins, electrolytes, flavors, colorants, pectic acids and their salts, alginic acids and their salts, organic acids, protective colloidal thickeners, pH regulators, stabilizers, preservatives, glycerin, alcohol, and carbonating agents used in carbonated beverages. In addition, the health functional food according to the present disclosure may contain fruit flesh for the production of natural fruit juice, fruit juice drinks, and vegetable drinks. These ingredients can be used independently or in combination with each other. A ratio of these additives is not limited, but is generally selected in the range of 0.01 to 0.1 parts by weight based on 100 parts by weight of the health functional food according to the present disclosure.

In another aspect, the present disclosure provides a method for improving liver health, comprising administering to a subject a composition comprising an effective amount of Gallocatechin Gallate (GCG) and Isoquercitrin (IQC).

In another aspect, the present disclosure provides a method for preventing, improving, or treating a liver disease, the method comprising administering to a subject a composition comprising an effective amount of Gallocatechin Gallate (GCG) and Isoquercitrin (IQC).

In still another aspect, the present disclosure provides a use of Gallocatechin Gallate (GCG) and Isoquercitrin (IQC) for preparing the composition for improving liver health.

In more still another aspect, the present disclosure provides Gallocatechin Gallate (GCG) and Isoquercitrin (IQC) for improving liver health.

In furthermore still another aspect, the present disclosure provides a non-therapeutic use of Gallocatechin Gallate (GCG) and Isoquercitrin (IQC) for improving liver health.

Hereinafter, the constitutions and effects of the present disclosure will be described in more detail through Examples. However, since the following Examples are provided only for illustrative purposes to aid understanding of the present disclosure, the scope and range of the present disclosure should not be limited by them.

### EXAMPLES

### Example 1

Comparison of an effect of inhibiting lipid accumulation in liver cells by treatment with a mixture (Adiphenon) of Gallocatechin Gallate (GCG) and Isoquercitrin (IQC)

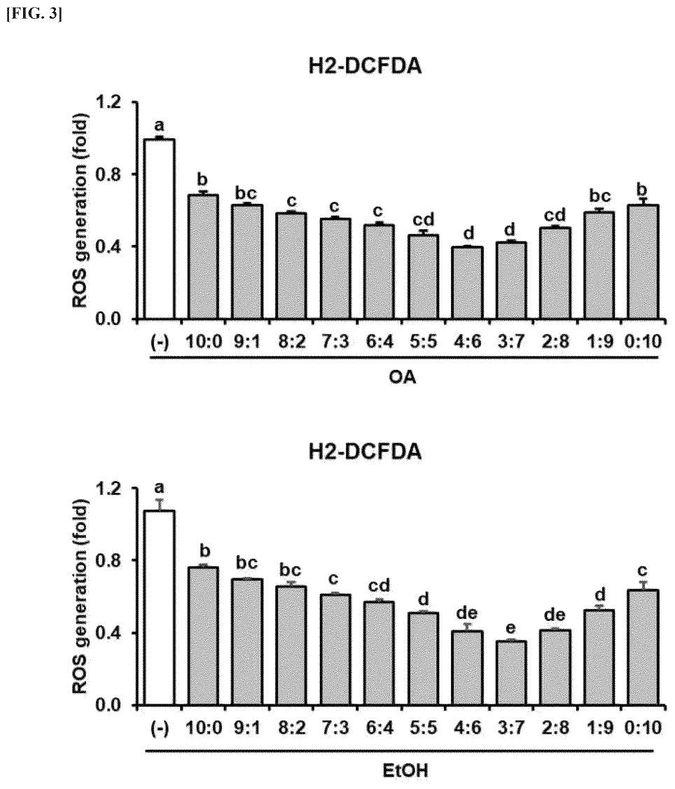

Fatty liver is literally a symptom of excessive accumulation of neutral lipids in a liver tissue (liver cells). It was investigated whether a mixture (Adiphenon) of Gallocatechin Gallate (GCG) and Isoquercitrin (IQC) can inhibit lipid accumulation in the liver cells. Specifically, the HepG2 human liver cancer cell line purchased from ATCC was cultured in a Roswell Park Memorial Institure-1640 medium (Sigma Aldrich) containing fetal bovine serum (Gibco) and 1% penicillin/streptomycin (P/S; Sigma Aldrich). In order to confirm an effect of improving fatty liver according to composition ratios of GCG and IQC, the fatty liver was induced by pretreating HepG2 cells with complexes having various ratios (GCG:IQC=10:0˜0:10) at a total concentration of 100 μg/ml for 2 hours, followed by treating the same with oleic acid (500 μM; Sigma Aldrich; FIG. 1) or ethanol (2 mM; Sigma Aldrich, FIG. 2) for 72 hours. Then, after the cells were washed twice with Phosphate Buffered Saline (PBS; Sigma Aldrich) and fixed in 10% formaldehyde (Sigma Aldrich) solution for 5 minutes, the accumulation of neutral lipids in the liver cells was confirmed by a nile-red (Sigma Aldrich; 300 nM) solution, which specifically stains neutral fats. An amount of the accumulated neutral fats was quantified using Tecan Infinite M200 Microplate Reader (ex 495 nm, em 585 nm).

As a result, as shown in FIGS. 1 and 2, when treated with various composition ratios of Adiphenon, the lipid accumulation in the liver cells was significantly reduced regardless of factors causing the fatty liver, and this reduction effect was found to be the best at GCG:IQC=3:7 to 4:6.

### Example 2

### Effect of Alleviating Oxidative Stress and Chronic Inflammatory Response in Liver Cells by Adiphenon Treatment

Alcohol generates a large amount of active oxygen during its decomposition process, and excessive fat intake increases a concentration of free fatty acids. The intrinsic immune response that protects cells from external harmful substances mainly begins with the binding of TLR4 and lipopolysaccharide, and TLR4 secretes various inflammatory factors through signaling systems such as NF-κB and AP-1. However, because TLR4 recognizes not only lipopolysaccharide, which is endotoxin, but also free fatty acids, a presence of excessive free fatty acids causes unwanted immune responses (e.g., chronic inflammation) even in the absence of external harmful substances. Since prolonged immune response is a major cause of secondary liver diseases such as cirrhosis, liver fibrosis, and liver cancer, the alleviation of oxidative stress and inflammatory response can help prevent and improve a fatty liver and related liver diseases.

Accordingly, an additional experiment was performed to determine whether Adiphenon could improve the oxidative stress and the inflammatory response caused during development of the fatty liver. Specifically, HepG2 cells were pretreated with various composition ratios of Adiphenon at a concentration of 100 μg/ml for 2 hours, and then treated with oleic acid (500 μM) or ethanol (2 mM) for 24 hours. After the same set of experiments was prepared in duplicate, some of the cells were washed twice with PBS as in Example 1, and were fixed with 10% formaldehyde. Thereafter, the cells were washed twice more with PBS to remove residual formaldehyde, and treated with H₂-DCFDA (20 μM; Invitrogen by Thermo Fisher Scientific), which can detect active oxygen, for 30 minutes. An amount of active oxygen produced was observed using a Tecan Infinite M200 Pro Microplate Reader (ex 492 nm, em 517 nm).

The remaining set of experiments was used for mRNA expression analysis so as to observe the inflammatory response. After RNA was extracted using TaKaRa MiniBEST Universal RNA Extraction Kit (Takara Bio), it was quantified using NanoQuant supplementary equipment of Tecan Infinite M200 (Tecan). Thereafter, cDNA was synthesized from the same amount (1 mg) of RNA using RevertAid 1st-strand cDNA Synthesis Kit (Thermo Fisher Scientific). In order to observe expression of genes involved in the inflammatory response, the coding sequence of the target gene was used as a template and NCBI primer BLAST (https://www.ncbi.nlm.nih.gov/tools/primer-blast/) service utilized to design and produce (Bioneer) primers. Expression of the corresponding genes was observed using the synthesized cDNA and primers by a CFX96 thermocycler (Bio-Rad).

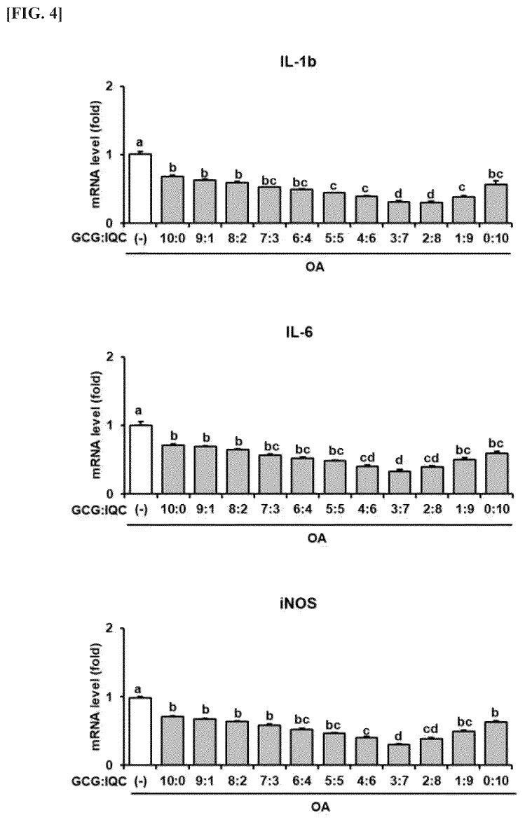

As a result, as shown in FIGS. 3, 4, and 5, effects of the active oxygen and the inflammatory response were confirmed by Adiphenon of various composition ratios, and these effects were found to be best at a ratio of 3:7 to 4:6.

### Example 3

### Comparison of Effects Depending on the Ratios of Adiphenon

There are various causes of fatty liver such as alcoholic fatty liver caused by excessive drinking and non-alcoholic fatty liver caused by excessive nutritional intake in dietary life. Regardless of the trigger, since the fatty liver involves oxidative stress and chronic inflammation during its development, oleic acid that causes excessive lipid accumulation within the cells was used from future examples. In order to find an optimal ratio that shows the best synergy for each of composition ratios of Adiphenon, the experiment of Example 1 was repeated after various treatments were performed at ratios of GCG:IQC=4:6 (about 1:1.5) to 2:8 (about 1:4).

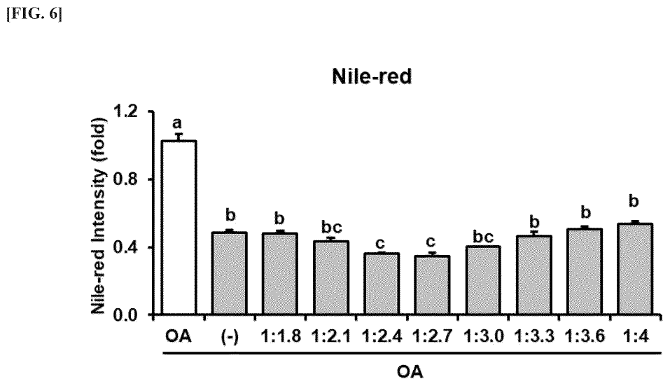

As a result, as shown in FIG. 6, the best lipid accumulation inhibition effect was observed when the ratios of GCG:IQC were between 1:2.4 and 1:2.7.

What is claimed is:

1. A method for improving liver health, comprising administering an effective amount of Gallocatechin Gallate (GCG) and Isoquercitrin (IQC) to a subject in need thereof.

2. The method according to claim 1, wherein the method is a method for preventing, improving, or treating a liver disease.

3. The method according to claim 2, wherein the liver disease is at least one selected from the group consisting of hepatitis, fatty liver, cirrhosis, and liver cancer.

4. The method according to claim 2, wherein the liver disease is an alcoholic liver disease.

5. The method according to claim 1, wherein the Gallocatechin Gallate (GCG) and Isoquercitrin (IQC) are contained in a weight ratio of 9:1 to 1:9.

6. The method according to claim 1, wherein the subject needs to reduce oxidative stress or inflammation in liver cells.

7. The method according to claim 6, wherein the subject needs to reduce expression of one or more genes selected from the group consisting of IL-1b, IL-6, and iNOS.

8. The method according to claim 1, wherein the subject needs to inhibit lipid accumulation in liver cells.
